(19) United States
(12) Patent Application Publication
(10) Pub. No.: US 2021/0330876 A1
(43) Pub. Date: Oct. 28, 2021

(54) ASPIRATOR HEAD ADAPTOR

(71) Applicant: Eir on the Side of Health, Simpsonville, SC (US)

(72) Inventors: Kacey Lentz, Simpsonville, SC (US); Ashley Perry, El Dorado Hills, CA (US); Haley Thorn, Indianapolis, IN (US)

(21) Appl. No.: 17/241,307

(22) Filed: Apr. 27, 2021

Related U.S. Application Data
(60) Provisional application No. 63/016,408, filed on Apr. 28, 2020.

(51) Int. Cl. A61M 1/00

(52) U.S. Cl. CPC A61M 1/71; A61M 2210/1433; A61M 2210/1021; A61M 2202/0413

(57) ABSTRACT

A suction head adaptor suitable for collecting fluid and blood during a surgical operation is provided. The suction head adaptor includes a first end and a second end opposite from the first end. The suction head adaptor may have a conical structure at the first end with a first aperture positioned at a center of the conical structure. A tube extends from the first aperture toward the second end and has a second aperture at the second end, allowing fluid to flow from the first aperture to the second aperture through the tube. An absorbent material such as a gauze can be provided within the conical structure. A splash guard can be provided on the tube to prevent splashing during suction, and ridges can be provided on the tube at the second end to allow the suction head adaptor to be inserted into a suction tube connected to an aspirator.

### CROSS-REFERENCE TO RELATED APPLICATIONS

This application claims priority to U.S. Provisional Patent Application Ser. No. 63/016,408, filed on Apr. 28, 2020, entitled “Aspirator Head Adaptor”, the entire disclosure of which is incorporated herein by reference.

### BACKGROUND OF THE INVENTION

Caesarean deliveries account for 31.9% of all deliveries in the US. While the average blood loss for a vaginal birth is approximately 500 cc, the average blood loss with a Caesarean delivery is about twice that amount.

During a Caesarean procedure, doctors cut through large blood vessels in order to open the wall of the uterus to gain access to the baby. Because of the large quantity of blood introduced into the surgical field, doctors utilize various blood collection methods such as suctions, surgical sponges, etc. These methods are typically used to clean the surgical site of blood and other fluids such as amniotic fluid and saline.

Surgical sponges are the only method to efficiently clean surgical sites. Suction is best utilized when dealing with pooled blood. Because of multiple locations for blood collection, total blood loss is visually estimated by the doctor.

Even though doctors have become reasonably accurate at this estimation, the various methods of blood collection make it difficult to accurately know the true amount of blood loss during the procedure. Out of 23,486 women who underwent primary Caesarean delivery, 3.2% were transfused with at least 2 to 3 units of blood. Even if transfusions are not necessary, a large number of units of blood are reserved and made available in case an emergency occurs. This leads to a large waste of financial, laboratory and blood bank resources.

Even in the US, there are limited stocks of fresh or stored blood. Therefore, preparing for transfusions at the end of procedures can cost up to $218 for each unit of red blood cells, which does not account for transportation or inflation due to the lack of supplies.

This problem can be exacerbated in developing countries with an even lower availability of blood and blood products. Thus, there is a need for a more efficient method of determining the total amount of blood loss during cesarean deliveries could lead to a more conservative usage of resources. Likewise, there is also a need for a method of collecting both wiped and suctioned blood more uniformly that could lead to a more informed decision by the doctor about the state of the patient and transfusion.

### BRIEF SUMMARY OF THE INVENTION

This disclosure generally relates to a suction head adaptor that allows a doctor to suction and wipe away blood using the same device.

In an embodiment, the adaptor can consist of a solid hemisphere exterior packed with absorbent material. The absorbent material can become initially saturated with blood, which may then be suctioned clean by the aspirator that it is attached to. Thus, the adaptor may be use continuously.

In some embodiments, a unique cavity may be provided that is filled with the absorbent material. In alternative embodiments, the cavity can be altered to include a splash guard in order to deflect any blood that may be lost during suctioning. The unique cavity can include an end that is placed into the suction/aspirator tube that includes various ridges for securing the device to the tube and collection container. Such a cavity can collect most, if not, all of the blood loss into the aspirator canister, thus reducing the locations in which blood is collected.

### BRIEF DESCRIPTION OF THE DRAWINGS

The accompanying drawings, which form a part of the specification and are to be read in conjunction like reference numerals are used to indicate like or similar parts in the various views.

FIGS. 1A-1D illustrate several views of a suction head adaptor according to an exemplary embodiment. Specifically, FIG. 1A illustrates a top perspective view; FIG. 1B illustrates a side view; FIG. 1C illustrates a top view; and FIG. 1D illustrates a bottom perspective view with a suction tube attached thereto.

FIGS. 2A-2D illustrate several views of a suction head adaptor according to another exemplary embodiment. Specifically, FIG. 2A illustrates a top perspective view; FIG. 2B illustrates a side view; FIG. 2C illustrates a top view; and FIG. 2D illustrates a bottom perspective view with a suction tube attached thereto.

FIG. 3 illustrates a perspective view of the suction head adaptor as shown in FIGS. 1A-1D, with an absorbent material inserted therein.

Before explaining the disclosed embodiment of the present invention in detail, it is to be understood that the invention is not limited in its application to the details of the particular arrangement shown, since the invention is capable of other embodiments. Exemplary embodiments are illustrated in referenced figures of the drawings. It is intended that the embodiments and figures disclosed herein are to be considered illustrative rather than limiting. Also, the terminology used herein is for the purpose of description and not of limitation.

### DETAILED DESCRIPTION

While this invention is susceptible of embodiments in many different forms, there are shown in the drawings and will be described in detail herein specific embodiments with the understanding that the present disclosure is an exemplification of the principles of the invention. It is not intended to limit the invention to the specific illustrated embodiments. The features of the invention disclosed herein in the description, drawings, and claims can be significant, both individually and in any desired combinations, for the operation of the invention in its various embodiments. Features from one embodiment can be used in other embodiments of the invention.

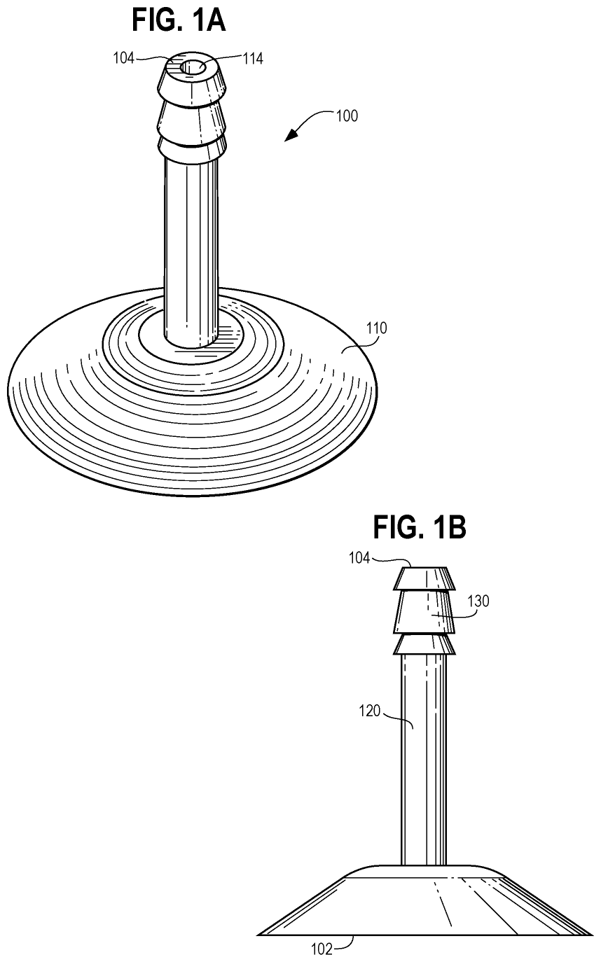

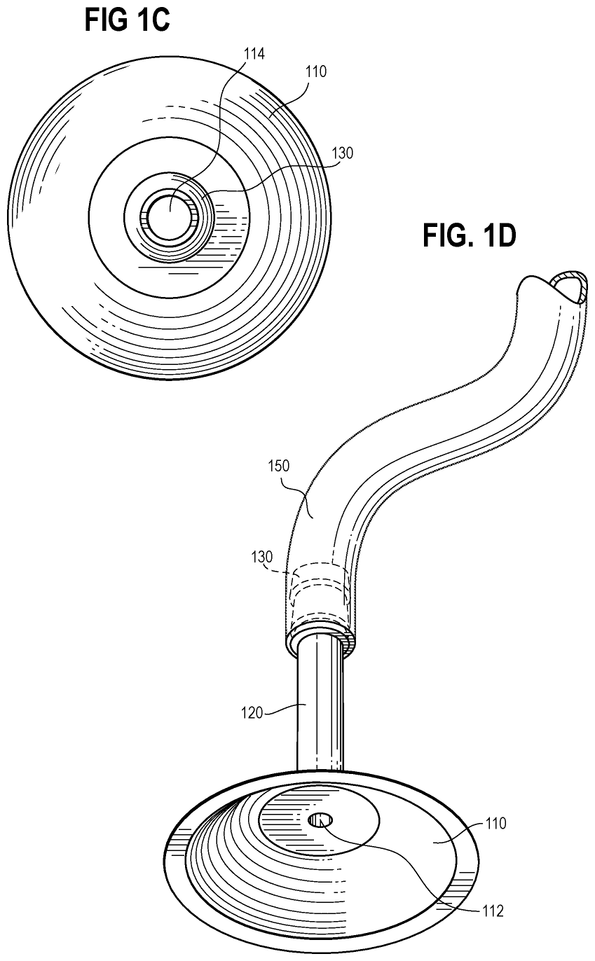

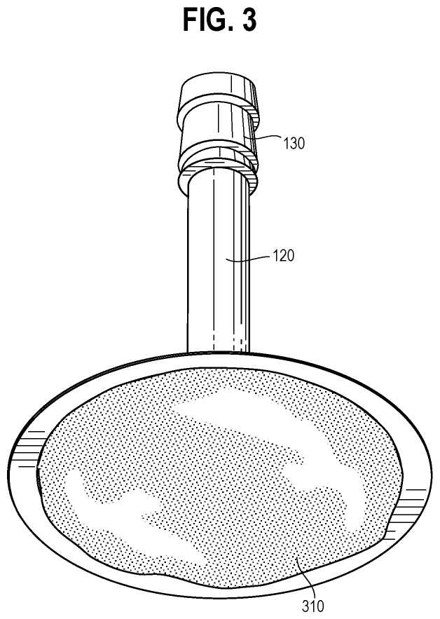

FIGS. 1-3 illustrate a suction head adaptor according to various embodiments. Referring first to FIGS. 1A, 1B, 1C, and 1D, a suction head adaptor 100 is provided according to an embodiment described herein. In such embodiment, where a splash guard that is described and included in embodiments described below is omitted, better mobility can be achieved. A first end 102 of the suction head adaptor 100, which may be defined by a rounded, conical-shaped side, can be filled with an absorbent substance. A second end 104 opposite the first end 102 can be inserted into a suction tube 150. The first end 102 of the suction head adaptor can be filled with an absorbent material 310, such as a gauze, as illustrated in FIG. 3 and described below.

Referring specifically to FIG. 1D, a conical structure 110 can surround the absorbent material used to wipe away and suction fluids from an area by putting the contained absorbent material in direct contact with the desired area. The conical structure 110 may guide blood captured by the absorbent material to an aperture 112 at the center of the conical structure 110, where it may then be suctioned up into a tube 120. The tube 120 can be rigid and generally cylindrical in shape. Blood and fluids collected from the first end 102 of the suction head adaptor 100 can travel through the tube 120 before entering the suction tube 150 connected to an aspirator (not illustrated) through a second aperture 114 provided at the second end 104. In some embodiments, ridges 130 can be provided on the tube 120 to secure the suction head adaptor 100 to the suction tube 150 of the aspirator without slipping.

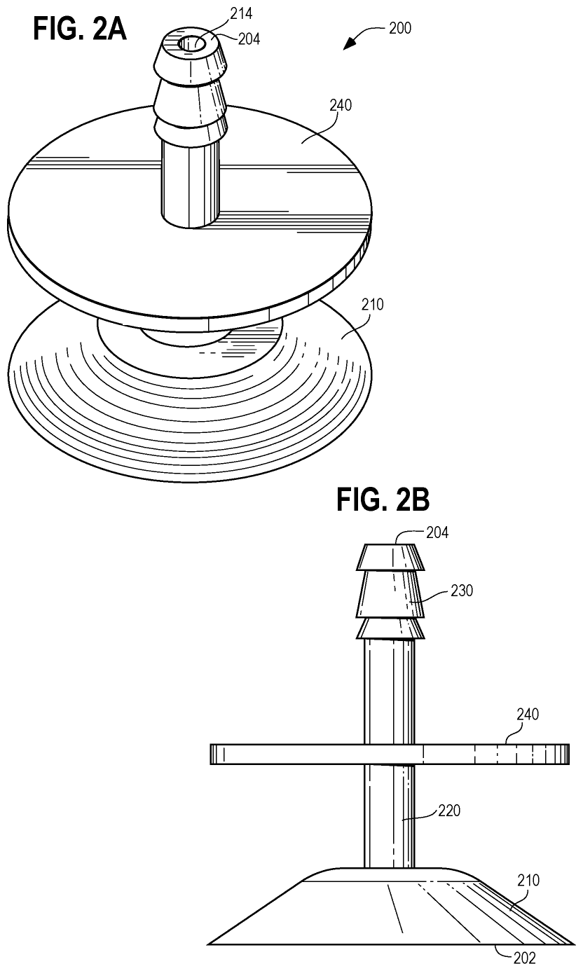

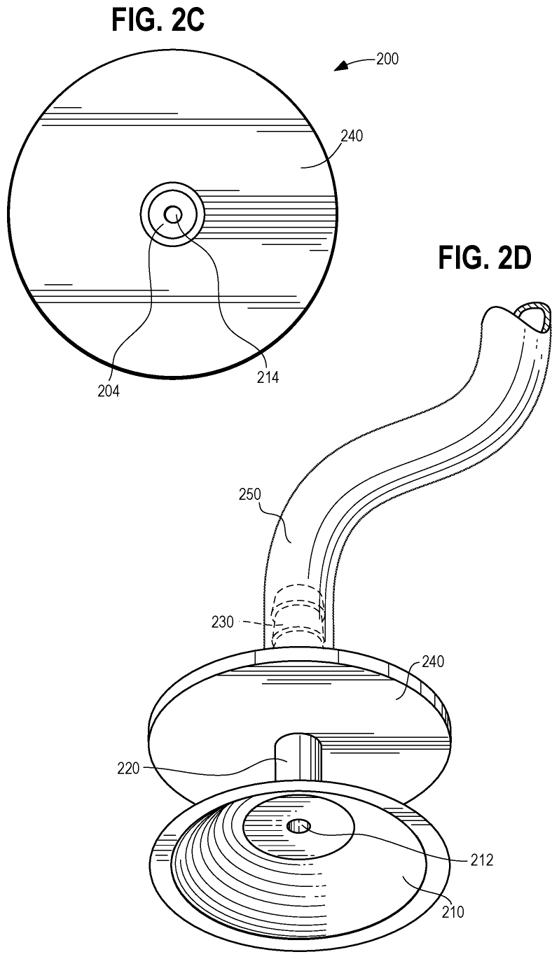

FIGS. 2A, 2B, 2C, and 2D illustrate an alternative suction head adaptor 200 according to another embodiment, the suction head adapter 200 including a splash guard 240. The splash guard 240 may better protect a suction tube 250 of an aspirator (not illustrated) from having blood or other fluid sprayed onto it during suction. Similar to the suction head adaptor 100 of FIGS. 1A-1D, a first end 202 of the suction head adaptor 200, defined by a rounded, conical side, can be filled with an absorbent substance, and a second end 204 can be inserted into the suction tube 250.

Referring specifically to FIG. 2D, a conical structure 210 can surround the absorbent material used to wipe away and suction fluids from an area by putting the contained absorbent material in direct contact with the desired area and guides blood captured by the absorbent material to the aperture 212 at the center to be suctioned up into a tube 220. The tube 220 can be rigid and cylindrical in shape. Blood and fluids collected from the first end 202 of the suction head adaptor 200 can travel through the tube 220 before entering the suction tube 250 connected to the aspirator through the aperture 214 as shown in FIG. 2D. In some embodiments, ridges 230 can be provided on the tube 220 to secure the suction head adaptor 200 into the suction tube 250 of the aspirator without slipping or becoming unattached. The splash guard 240 can take the form of a rigid disc extending from the tube 220. The splash guard 240 can be used to reduce fluid splatter from contaminating the end of the suction tube 250 connected to the suction head adaptor 200.

Turning to FIG. 3, absorbent material 310 (such as surgical gauze) can be placed inside the conical structure 110, 210 of the suction head adaptor 100, 200 provided in FIGS. 1A-1D and 2A-2D. The absorbent material 310 can be used to wipe away fluids. In some embodiments, the absorbent material 310 can be pleated to allow pooled blood to be suctioned up more directly rather than be absorbed then suctioned. Small quantities of fluid that are usually wiped away can be absorbed by the absorbent material 310 before being suctioned away, thus allowing more fluid to be collected.

Referring to FIGS. 1-3, in operation, the suction head adaptor 100, 200 can be attached to the suction tube 150, 250 by inserting the second end 104, 204, respectively, into the suction tube 150, 250 that is connected to the aspirator. Once attached, the suction head adaptor 100, 200 can be used to suction or wipe up fluids from the surgical area during operation of the aspirator.

The absorbent material 310 within the first end 102, 202, preferably first absorbs fluid upon contact therewith. The conical structure 110, 210 surrounding the absorbent material 310 may be structured to taper inwardly as it approaches the second end 104, 204 such that collected fluid can be directed to the aperture 112, 212 at the center of the first end 102, 202 and suctioned up into the tube 120, 220 extending from the aperture 112, 212, and into the suction tube 150, 250 connected to the aspirator through the aperture 114, 214. In some embodiments, the splash guard 240 can be provided, so that fluid splattering because of the suction can be blocked, fully or partially, from spraying onto the suction tube 150, 250 or the surgeon using the suction head adaptor 200.

An aspirator system may simultaneously suction the absorbent material 310 dry, thus allowing the suction head adaptor 100, 200 to be reused throughout a procedure, which allows for fluid collection during the procedure to be more centralized to the aspirator canister. This preferably results in a more accurate estimate of blood and general fluid loss. In some embodiments, the suction head adaptor 100, 200 can be used in conjunction with a multi-sensor blood loss monitor system such as those disclosed in to U.S. Provisional Patent Application Ser. No. 63/016,412, filed on Apr. 28, 2020, entitled “MULTI-SENSOR IN-REAL-TIME BLOOD LOSS MONITOR,” the content of which is hereby incorporated by reference in its entirety.

From the foregoing, it will be seen that this invention is one well-adapted to attain all the ends and objects hereinabove set forth together with other advantages which are obvious, and which are inherent to the structure. It will be understood that certain features and sub combinations are of utility and may be employed without reference to other features and sub combinations. This is contemplated by and is within the scope of the claims. Since many possible embodiments of the invention may be made without departing from the scope; thereof, it is also to be understood

The constructions described above and illustrated in the drawings are presented by way of example only and are not intended to limit the concepts and principles of the present invention. Thus, there has been shown and described several embodiments of a novel invention. As is evident from the foregoing description, certain aspects of the present invention are not limited by the particular details of the examples illustrated herein, and it is therefore contemplated that other modifications and applications, or equivalents thereof, will occur to those skilled in the art. The terms “having” and “including” and similar terms as used in the foregoing specification are used in the sense of “optional” or “may include” and not as “required”. Many changes, modifications, variations and other uses and applications of the present construction will, however, become apparent to those skilled in the art after considering the specification and the accompanying drawings. All such changes, modifications, variations and other uses and applications which do not depart from the spirit and scope of the invention are deemed to be covered by the invention which is limited only by the claims which follow.

What is claimed is:

1. A suction head adaptor for drawing fluids from a surgical site, the suction head comprising:
a conical structure positioned at a first end of the suction head adaptor, the conical structure including a first aperture positioned at a center of the conical structure;
a tube extending from the first aperture toward a second end of the suction head adaptor, the second end opposite from the first end; and
an absorbent material received within the conical structure.

2. The suction head adaptor of claim 1, wherein the suction head adaptor further includes one or more ridges on an external surface of the tube proximal to the second end of the suction head adaptor, and wherein the the one or more ridges are configured to secure the suction head adaptor within a suction tube.

3. The suction head adaptor of claim 1, wherein the absorbent material is a gauze.

4. The suction head adaptor of claim 1, the suction head adaptor further comprising a splash guard extending radially outwardly from the tube.

5. The suction head adaptor of claim 4, wherein the splash guard is a rigid disc positioned between the conical structure and the second end of the suction head adaptor.

6. The suction head adaptor of claim 1, wherein the tube comprises a second aperture at the second end of suction head adaptor to permit a fluid to flow from the first aperture to the second aperture through the tube.

7. A suction head adaptor for drawing fluids from a surgical site, the suction head comprising:
a first end and a second end;
a structure positioned at the first end, the structure including a first aperture positioned at a center of the structure, and the structure tapering inwardly from the first end to the second end to form a cone shape;
a tube extending from the first aperture toward the second end of the suction head adaptor, the tube including a second aperture at the second end of suction head adaptor to permit a fluid to flow from the first aperture to the second aperture through the tube;
an absorbent material inserted into the structure; and
a splash guard extending radially outwardly from the tube.

8. The suction head adaptor of claim 7, wherein the absorbent material is a gauze.

9. The suction head adaptor of claim 7, wherein one or more ridges are provided on an external surface of the tube proximal to the second end of the suction head adaptor.

10. The suction head adaptor of claim 9, wherein the one or more ridges are configured to hold the suction head adaptor within a suction tube.

11. The suction head adaptor of claim 7, wherein the splash guard includes a hole member through which the tube is received.

12. The suction head adaptor of claim 7, wherein the splash guard is circular in shape.
